(19) United States
(12) Patent Application Publication
(10) Pub. No.: US 2025/0114431 A1
(43) Pub. Date: Apr. 10, 2025

(54) COMPOUND, COMPOSITION AND METHODS FOR LOWERING CIRCULATING GLUCOSE

(71) Applicant: THE HONG KONG POLYTECHNIC UNIVERSITY, Hong Kong (CN)

(72) Inventors: Chi Ming WONG, Hong Kong (CN); Tak Ho LO, Hong Kong (CN)

(21) Appl. No.: 18/483,597

(22) Filed: Oct. 10, 2023

(51) Int. Cl. A61K 38/17; A61P 3/10

(52) U.S. Cl. CPC A61K 38/1709; A61P 3/10

(57) ABSTRACT

The present invention provides a compound or derivatives thereof capable of lowering circulating glucose in a subject. The compound is a recombinant placenta-specific protein 9 (Plac9) or derivatives thereof that can enhance glucose uptake by liver cells or hepatocytes in an insulin-independent manner, thereby lowering the circulating glucose. The present invention also provides a composition including an effective amount of the compound or derivatives thereof and a method for treating diseases or conditions associated with or arising from high circulating glucose such as diabetes mellitus.

### REFERENCE TO SEQUENCE DISCLOSURE

A sequence listing file with a file name “P25422US00_sequence_listing.xml” in ST.26 XML file format having a file size of 8 KB created on Sep. 13, 2023 is incorporated herein by reference in its entirety

### TECHNICAL FIELD

The present invention relates to a compound, composition, and methods for lowering circulating glucose level in a subject.

### BACKGROUND

Diabetes has been a global health issue and becoming more significant in wealthy and relatively more developed countries. Impaired insulin production and utilization effectiveness are the major causes of diabetes. Over time, the high glucose concentration in diabetes patients leads to various complications including heart attack, stroke, blindness, kidney failure, and lower limb amputation, imposing significant healthcare burdens on society. Unfortunately, there is no complete cure for diabetes. Mild to serious diabetes patients need to keep taking drugs to maintain their blood glucose levels. Diabetic drugs are mainly classified by their actions and/or routes of administration. Among them, protein therapeutics have more specific therapeutic actions, lower side effect, and are usually well-tolerated than others such as small-molecule drugs. One example is glucagon-like peptide (GLP-1) agonist, Liraglutide (NN211, marketed as Victoza). Another example is Exenatide (marketed as Byetta) which has been used clinically for treating diabetes. The most recent one is the FDA-approved oral GLP-1 treatment, Semaglutide, for type 2 diabetes in 2019.

While insulin secretion stimulants can be effective in treating some types of diabetes, there are still some concerns associated with their use. Some studies suggested that primary insulin hypersecretion can lead to beta cell dysfunction, as seen in patients with persistent hyperinsulinemic hypoglycemia of infancy (PHHI) who have genetic mutations causing overt insulin hypersecretion. In fact, a recent study suggested that this autosomal dominant mutation in sulfonylurea receptor-1 (SUR1) causing congenital hyperinsulinism followed by insulin deficient diabetes is considered as a new genetic subclass of type 2 diabetes with similar phenotypes of glucose intolerance, beta cell dysfunction, and hyperglycemia. Some in vitro studies also supported this finding, showing that chronic treatment of beta cells with agents that stimulate insulin secretion, such as sulfonylureas, can cause cell death via apoptosis. On the other hand, allowing beta cells to rest by reducing insulin secretion could be beneficial and protect against glucose toxicity and oxidative stress. That explains why the frequency of failure of current first- or second-line therapy for treatment of diabetes at 5 years is up to 15-34%. Alternative approaches to treating diabetes that do not rely solely on insulin secretion stimulants.

Compared to type 2 diabetes, type 1 diabetes is a chronic autoimmune disease affecting more people globally. This disease occurs when the body's immune system mistakenly attacks and destroys insulin-producing cells in the pancreas. As a result, people with type 1 diabetes are unable to produce enough insulin to regulate their blood sugar levels. This means that agents that stimulate insulin secretion cannot be used to treat type 1 diabetes. Treating type 1 diabetes is challenging because it requires constant monitoring of blood sugar levels and insulin injections or the use of an insulin pump. Even with careful management, people with type 1 diabetes are still at risk for long-term complications such as heart disease, kidney failure, and blindness. Finding effective treatments for improving the quality of life of these type 1 diabetes patients is crucial.

One of the most recent advancements in the field of type 1 diabetes treatment is the FDA-approved Tzield (teplizumab-mzwv) injection, which can delay the onset of stage 3 (clinically symptomatic) type 1 diabetes in adults and pediatric patients aged eight years and older who currently have stage 2 (glucose intolerance or dysglycemia) type 1 diabetes. Tzield binds to certain immune system cells, delaying progression to stage 3 type 1 diabetes. It may deactivate immune cells that attack insulin-producing cells while increasing the proportion of cells that help moderate the immune response. However, Tzield is administered by intravenous infusion once daily for 14 consecutive days, and the most common side effects include decreased levels of certain white blood cells, rash, and headache. The use of Tzield comes with warnings and precautions, including premedication and monitoring for symptoms of Cytokine Release Syndrome, the risk of serious infections, decreased levels of lymphocytes, the risk of hypersensitivity reactions, the need to administer all age-appropriate vaccinations before starting Tzield, and avoiding concurrent use of live, inactivated and mRNA vaccines with Tzield.

Therefore, there is an unmet need for an effective drug for both type 1 and type 2 diabetes that eliminates or at least diminishes the disadvantages and problems described above.

### SUMMARY OF INVENTION

Accordingly, a first aspect of the present invention provides a compound comprising a protein, peptide, or any derivative thereof with functions of enhancing glucose uptake by liver cells or hepatocytes independent from insulin, thereby lowering the circulating glucose level in a subject.

A second aspect of the present invention provides a composition comprising an effective amount of the compound or any derivative thereof described in the first aspect for lowering circulating glucose level and treating diabetes in a subject.

A third aspect of the present invention provides a method for lowering circulating glucose level in a subject comprising administering an effective amount of the compound or derivative thereof described in the first aspect or the composition described in the second aspect to a subject in need thereof.

A fourth aspect of the present invention provides a method for treating diseases or conditions associated with or arising from hyperglycemia, glucose intolerance, or pancreatic beta cell dysfunction comprising administering an effective amount of the compound or derivatives thereof described in the first aspect or the composition described in the second aspect or third aspect to a subject in need thereof.

In certain embodiments, the compound is a recombinant protein or peptide represented by an amino acid sequence of SEQ ID NO: 1 or 2.

In certain embodiments, the compound is a recombinant placenta-specific protein 9 (Plac9).

In certain embodiments, the recombinant Plac9 is synthesized and purified from a bacterial expression system such as Escherichia coli (E. coli).

In certain embodiments, the compound is capable of improving glucose uptake by liver cells or hepatocytes in an insulin-independent manner, thereby lowering the circulating glucose level.

In certain embodiments, the compound or the composition can be administered via intravenous, intraperitoneal, or intramuscular injection, or via an oral or subcutaneous route.

In certain embodiments, the composition further comprises a pharmaceutically acceptable carrier, ester, or salt.

In certain embodiments, the derivative is a proteins or peptide having an amino acid sequence of SEQ ID NO: 3 or 4, or any protein or peptide with a sequence homology of at least 73% similarity to the amino acid sequence of SEQ ID NO: 1 or 2, or a nucleotide encoding any of said protein or peptide.

In certain embodiments, the diseases or conditions of the subject comprise diabetes mellitus comprising type 1 and type 2 diabetes, cardiovascular diseases comprising hypertension, heart attack, and stroke, blindness, kidney failure, and lower limb amputation.

In certain embodiments, the subject comprises human or non-human mammals.

This summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. Other aspects of the present invention are disclosed as illustrated by the embodiments hereinafter.

### BRIEF DESCRIPTION OF DRAWINGS

The appended drawings, where like reference numerals refer to identical or functionally similar elements, contain figures of certain embodiments to further illustrate and clarify the above and other aspects, advantages and features of the present invention. It will be appreciated that these drawings depict embodiments of the invention and are not intended to limit its scope. The invention will be described and explained with additional specificity and detail through the use of the accompanying drawings in which:

FIG. 1 shows a sequence alignment between human Plac9 (hPLAC9) and mouse Plac9 (mPLAC9), where symbol “*” represents identical amino acid residue; symbol “:” or “⋅” represents similar amino acid residue.

FIGS. 2A-2I show the efficacy of the recombinant Plac9 according to certain embodiments to improve the glucose tolerance in an obese mouse model induced by high fat diet (HFD), where the data in the curves or plots are expressed as mean±SEM; N.S.: not significant; ***p<0.001, **p<0.01, *p<0.05: FIG. 2A schematically depicts an experimental design of administering the recombinant Plac9 via intraperitoneal (i.p.) injection to the HFD-fed obese mice (n=8) at 2 mg/kg body weight (BW) once daily (q.d.) versus i.p. injection of phosphate-buffered saline (PBS) as a control over an 18-day time course; FIG. 2B shows a change in body weight measured every 3 days over the time course depicted in FIG. 2A; FIG. 2C shows a change in fasting blood glucose measured every 3 days over the time course depicted in FIG. 2A; FIG. 2D shows a change in feeding blood glucose measured every 3 days over the time course depicted in FIG. 2A; FIG. 2E shows a change in blood insulin at Day 0 and Day 15 during the time course depicted in FIG. 2A; FIG. 2F shows the result of glucose tolerance test (GTT) in terms of the blood glucose level at Day 0 of the time course depicted in FIG. 2A (left panel) and the area under the curve (AUC) (right panel); FIG. 2G shows the percentage change in blood glucose level as shown in FIG. 2F (left panel) and the area under the curve (AUC) (right panel); FIG. 2H shows the result of glucose tolerance test in terms of the blood glucose level at Day 15 of the time course depicted in FIG. 2A (left panel) and the area under the curve (AUC) (right panel); FIG. 2I shows the percentage change in blood glucose level as shown in FIG. 2H (left panel) and the area under the curve (AUC) (right panel).

FIGS. 3A-3I show the efficacy of the recombinant Plac9 according to certain embodiments to improve the glucose tolerance and also increase the endogenous insulin level in a diabetic mouse model induced by streptozotoxin (STZ), where the data in the curves or plots are expressed as mean±SEM; ***p<0.001. **p<0.01. *p<0.05: FIG. 3A schematically depicts an experimental design of administering the recombinant Plac9 via intraperitoneal (i.p.) injection to the STZ-induced diabetic mice (n=8) at (BW) once daily (q.d.) versus i.p. injection of phosphate-buffered saline (PBS) as a control over an 18-day time course; FIG. 3B shows a change in body weight of Plac9-treated mice versus the control mice measured every 3 days over the time course depicted in FIG. 3A; FIG. 3C shows a change in fasting blood glucose of Plac9-treated mice versus the control mice measured every 3 days over the time course depicted in FIG. 3A; FIG. 3D shows a change in feeding blood glucose of Plac9-treated mice versus the control mice measured every 3 days over the time course depicted in FIG. 3A; FIG. 3E shows a change in blood insulin of Plac9-treated mice versus the control mice at Day 0, Day 5 and Day 15 during the time course depicted in FIG. 3A; FIG. 3F shows the result of glucose tolerance test in terms of the blood glucose level of Plac9-treated mice versus the control mice at Day 0 of the time course depicted in FIG. 3A (left panel) and the area under the curve (AUC) (right panel); FIG. 3G shows the percentage change in blood glucose level as shown in FIG. 3F (left panel) and the area under the curve (AUC) (right panel); FIG. 3H shows the result of glucose tolerance test (GTT) in terms of the blood glucose level at Day 15 of the time course depicted in FIG. 3A (left panel) and the area under the curve (AUC) (right panel); FIG. 3I shows the percentage change in blood glucose level as shown in FIG. 3H (left panel) and the area under the curve (AUC) (right panel).

FIG. 4A-4W show that an adenovirus-mediated Plac9 expression alleviates blood glucose level in an HFD-induced obese mouse model, where the data in the curves or plots are expressed as mean±SEM; ***p<0.001, **p<0.01, *p<0.05: FIG. 4A schematically depicts an experimental design of introducing an AAV-mediated overexpression of Plac9 (treatment group) or GFP (control group) in an HFD-induced obese mouse model (n=8 in each group) at Week 6 over a 20-week time course; FIG. 4B shows a change in body weight of the treatment and control groups measured every 2 weeks after starting from Week 8 until end of the time course depicted in FIG. 4A; FIG. 4C shows a change in fasting blood glucose level of the treatment and control groups over the time course depicted in FIG. 4A; FIG. 4D shows a change in feeding blood glucose level of the treatment and control groups over the time course depicted in FIG. 4A; FIG. 4E shows the difference in circulating Plac9 levels of the treatment and control groups at Week 8 and Week 16 during the time course depicted in FIG. 4A; FIG. 4F shows the difference in circulating insulin levels of the treatment and control groups at Week 8 and Week 16 during the time course depicted in FIG. 4A; FIG. 4G shows the results of glucose tolerance test (GTT) in terms of blood glucose level of the treatment and control groups measured at Week 12 during the time course depicted in FIG. 4A (left panel) and the area under the curves (AUC) (right panel); FIG. 4H shows the percentage change in blood glucose level as shown in FIG. 4G (left panel) and the area under the curve (AUC) (right panel); FIG. 4I shows the results of insulin tolerance test (ITT) in terms of the blood glucose level of the treatment and control groups measured at Week 20 which is end of the time course depicted in FIG. 4A (left panel) and the area under the curves (AUC) (right panel); FIG. 4J shows the percentage change in blood glucose level as shown in FIG. 4I (left panel) and the area under the curve (AUC) (right panel); FIG. 4K shows the results of pyruvate tolerance test (K) in terms of the blood glucose level of the treatment and control groups measured at Week 16 during the time course depicted in FIG. 4A (left panel) and the area under the curves (AUC) (right panel); FIG. 4L shows the percentage change in blood glucose level as shown in FIG. 4K (left panel) and the area under the curve (AUC) (right panel); FIG. 4M shows fat mass level of the treatment and control groups measured at Week 20 which is the end of the time course depicted in FIG. 4A; FIG. 4N shows the percentage change in fat mass as shown in FIG. 4M; FIG. 4O shows lean mass level of the treatment and control groups measured at the end of the time course depicted in FIG. 4A; FIG. 4P shows the percentage change in lean mass as shown in FIG. 4O; FIG. 4Q shows circulating alanine aminotransferase (ALT) level of the treatment and control groups measured at the end of the time course depicted in FIG. 4A; FIG. 4R shows circulating aspartate aminotransferase (AST) level of the treatment and control groups measured at the end of the time course depicted in FIG. 4A; FIG. 4S shows circulating triglyceride level of the treatment and control groups measured at the end of the time course depicted in FIG. 4A; FIG. 4T shows circulating free fatty acid level of the treatment and control groups measured at the end of the time course depicted in FIG. 4A; FIG. 4U shows circulating cholesterol level of the treatment and control groups measured at the end of the time course depicted in FIG. 4A; FIG. 4V shows circulating high density lipoprotein (HDL) level of the treatment and control groups measured at the end of the time course depicted in FIG. 4A; FIG. 4W shows circulating low density lipoprotein (LDL) level of the treatment and control groups measured at the end of the time course depicted in FIG. 4A.

FIGS. 5A-5C show that Plac9 enhances glucose uptake by liver cells or hepatocytes in an insulin-independent manner, in which the data are expressed as mean±SEM; n=8: FIG. 5A shows the results of glucose stimulated insulin secretion assay for MIN6 pancreatic beta cells treated with PBS (negative control), Exendin-4 (100 nM) (positive control) and recombinant Plac9 at different concentrations (1000, 100, 10 nm) (n=8); FIG. 5B shows the effect of recombinant Plac9 at different concentrations (1, 0.5, 0.25, 0.125 μM) on glucose uptake in primary hepatocytes in the presence (10 nM) or absence of insulin by using a glucose uptake assay to measure the level of 2-deoxyglucose-6-phosphate (2DG6P); FIG. 5C shows the effect of recombinant Plac9 at different concentrations (1, 0.5, 0.25 μM) on glucose uptake in HepG2 cells in the presence (10 nM) or absence of insulin by using the same glucose uptake assay as in FIG. 5B.

Skilled artisans will appreciate that elements in the figures are illustrated for simplicity and clarity and have not necessarily been depicted to scale.

### DETAILED DESCRIPTION OF THE INVENTION

It will be apparent to those skilled in the art that modifications, including additions and/or substitutions, may be made without departing from the scope and spirit of the invention. Specific details may be omitted so as not to obscure the invention; however, the disclosure is written to enable one skilled in the art to practice the teachings herein without unduc experimentation.

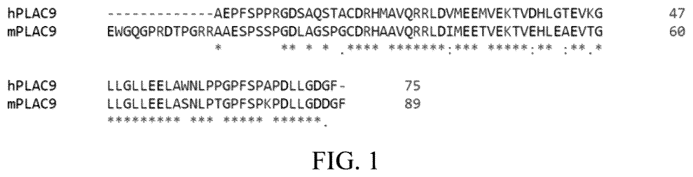

The present invention relates to a compound for enhancing glucose uptake by liver cells or hepatocytes in an insulin-independent manner. The present compound is derived from placenta-specific protein 9 (Plac9) with the signal peptide truncated before use. Plac9 is an adipokine that is mainly expressed in pre-adipocytes. The wild-type Plac9 of human origin (hPLAC9) has a molecular weight of 10,309 Da, whereas the equivalent of mouse origin (mPLAC9) has a molecular weight of 11,275 Da. Sequence alignment between the amino acid sequences of hPLAC9 and mPLAC9 is shown in FIG. 1A. From the result in FIG. 1A, in the absence of their respective signal peptide, there is about 78% similarity between the two Plac9 sequences. When the amino acid sequence of hPLAC9 with the signal peptide represented by SEQ ID NO: 5 is compared with that of two other isoforms (placenta-specific 9, isoform CRA_b and placenta associated 9 represented by SEQ ID NOs: 3 & 4, respectively), it shows that the main chain of Plac9 is highly conserved between its wild-type and isoforms of human origin, and referring to the alignment shown in FIG. 1A, it is also highly conversed between human and mouse. As a result, the main chain of Plac9 is expressed in a bacterial expression system or a coding sequence thereof is introduced into a viral vector such as adenovirus to overexpress the same in the host.

In certain embodiments, the protein or its derivatives can be synthesized by any known methods including a solid-phase peptide synthesis described by Merrifield, J. M. (Chem. Soc. 85:2149 (1962)), Stewart a Young (Solid Phase Peptide Synthesis (Freeman, San Francisco, 1969), pages 27-66), or purification from cells overexpressing Plac9 by recombinant DNA technologies (Maniatis, T, et al., Molecular Biology: A Laboratory Manual, Cold Spring Harbor, New York (1982)), which are incorporated by reference herein. Other known methods include protein fragmentation of the naturally occurring protein sequence, if appropriate.

As used herein, the term “isolated” in connection with a compound described herein means the compound is not in a cell or organism and the compound is separated from some or all of the components that typically accompany it in a cell or organism.

As used herein, the term “substantially pure” in connection with a sample of a compound described herein means the sample contains at least 60% by weight of the compound. In certain embodiments, the sample contains at least 70% by weight of the compound; at least 75% by weight of the compound; at least 80% by weight of the compound; at least 85% by weight of the compound; at least 90% by weight of the compound; at least 95% by weight of the compound; or at least 98% by weight of the compound.

The term “derivatives” or alike of a protein described herein may refer to a compound, molecule, or moiety sharing substantial homology with their naturally occurring form, with a similarly sized fragment to that of the naturally occurring form, functionally equivalent, similar, or even superior to a glucoregulatory hormone, and/or having higher insulinotropic and blood glucose lowering activities than those of the naturally occurring form. For instance, one or more residues in the amino acid sequence of the naturally occurring Plac9 can be substituted with other residue(s), e.g., lysine is substituted with arginine, or isoleucine is substituted with leucine, or tyrosine is substituted with phenylalanine. These homologs may have at least 95% homology to their naturally occurring peptide, which are still expected to have similar or even substantially equivalent efficacy. In some cases, having the same activity exerted on target cells or tissue, one or more of these derivatives may only be in a concentration of 10-11M, which is about 10-fold lower in concentration than that of their naturally occurring form.

As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In certain embodiments, organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, iron, zinc, copper, manganese, aluminum, and the like. Further pharmaceutically acceptable salts include, when appropriate, non-toxic ammonium, quaternary ammonium, and amine cations formed using counterions, such as halide, hydroxide, carboxylate, sulfate, phosphate, nitrate, lower alkyl sulfonate, and aryl sulfonate. Organic bases from which salts can be derived include, for example, primary, secondary, and tertiary amines, substituted amines including naturally occurring substituted amines, cyclic amines, basic ion exchange resins, and the like, such as isopropylamine, trimethylamine, diethylamine, triethylamine, tripropylamine, and ethanolamine. In certain embodiments, the pharmaceutically acceptable base addition salt is chosen from ammonium, potassium, sodium, calcium, and magnesium salts.

The term “pharmaceutically acceptable salts” described herein may also refer to the relatively non-toxic, inorganic and organic acid addition salts of the glucoregulatory compound of the present disclosure. These salts can be prepared in situ in the administration vehicle or the dosage form manufacturing process, or by separately reacting a purified glucoregulatory compound of the invention in its free base form with a suitable organic or inorganic acid, and isolating the salt thus formed during subsequent purification. Representative salts include the bromide, chloride, sulfate, bisulfate, carbonate, bicarbonate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts and the like.

The pharmaceutically acceptable salts of the glucoregulatory compound of the present disclosure includes the conventional nontoxic salts or quaternary ammonium salts of the compound, e.g., from nontoxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

The compound of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of the compound of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like.

The present invention further provides a use of the protein or its derivatives in manufacturing or preparation of a pharmaceutical composition (or a medicament) for treating a disease in a subject, where the diseases include hyperglycemia, type 1 diabetes or type 2 diabetes mellitus.

The present invention additionally provides a method for treating a disease associated with insulin-deficiency or failure in glucose uptake by certain cells in a subject comprising administering the composition comprising an effective amount of protein or its derivatives to the subject in need thereof. The administration is not limited to oral, but also includes intravenous, intraperitoneal, intramuscular, and subcutaneous routes of administration.

The pharmaceutical composition of the present disclosure may be specially formulated for administration in liquid form, including those adapted for the following: parenteral administration, for example, by intravenous as, for example, a sterile solution or suspension.

In exemplary embodiments, the subject includes human and non-human mammals.

Alternatively, the protein or its derivatives may also be delivered to said subject with an expression vector including an adenovirus-mediated expression vector containing an encoding sequence of the protein or its derivatives to be expressed in host cells or target tissues of the subject.

In certain embodiments, the proteins (or derivatives) of Plac9, and/or nucleic acids that encoding the Plac9, may be used as therapeutic compositions. Such therapeutic compositions may consist solely of the proteins (or derivatives), or with the nucleic acid encoding the same, although, preferably, the compositions will contain the peptides (or derivatives thereof) in admixture with a pharmaceutically acceptable carrier vehicle.

In certain embodiments, the composition comprising the Plac9 may be administered intravenously, intramuscularly, subcutaneously or orally at dosages in a range from about 1 μg/kg to 10 mg/kg body weight, or at a concentration sufficient to produce serum levels of 10⁻¹⁰ M to 10⁻¹¹ M, although a lower or higher dosage may be administered. Dosage is variable subject to various factors including, but not limited to, the severity of the condition of the subject in need thereof, for example, the severity of a patient's hyperglycemia, and upon such criteria as the patient's height, weight, sex, age, and medical history. The dose may also vary depending upon whether the composition of the invention is administered in what setting, e.g., a veterinary setting to a smaller animal or in a physician setting to a human subject.

For the purpose of parenteral administration, the composition comprising the Plac9 or its derivatives are preferably dissolved in distilled water and the pH-value is preferably adjusted to about 6 to 8. In certain embodiments, the composition is formulated in lyophilized form. In order to facilitate the lyophilization process, lactose may be added to the solution. Preferably, the solution is then filtered, sterilized, introduced into vials, and lyophilized. In a preferred embodiment, the composition is administered orally to a subject at the time of eating or shortly thereafter. The concentration of the Plac9 derivatives in these compositions, and especially the concentration of Plac9 whether oral or parenteral, may vary from 10⁻¹² M to 10⁻³ M.

Pharmaceutical compositions of the present invention suitable for parenteral administration comprise the glucoregulatory compound described herein in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain sugars (such as sucrose), alcohols, non-ionic surfactants (such as Tween 20), antioxidants, buffers, bacteriostats, chelating agents, solutes which render the formulation isotonic with the blood of the intended recipient or suspending or thickening agents.

Additional pharmaceutical methods may be employed to control the duration of action. The present composition may be formulated into a controlled release preparation which can be achieved by using certain polymers to complex or adsorb the Plac9 or its derivatives. The controlled release system may be enabled by selecting appropriate macromolecules (for example, polyesters, polyamino acids, polyvinyl pyrrolidone, ethylene vinylacetate, methylcellulose, carboxymethylcellulose, and protamine sulfate), the concentration of macromolecules, as well as the methods of incorporation in order to exert control release effect. Another possible method to control the duration of action of the composition is to incorporate the PLAC9 or its derivatives into certain particles made of a copolymeric material such as polyethylene vinylacetate copolymers. Alternatively, instead of incorporating the Plac9 or its derivatives into the copolymeric particles, it is possible to entrap which in microcapsules which are prepared, for example, by coacervation techniques, by interfacial polymerization with hydroxymethylcellulose or gelatin microcapsules and poly (methylmethacrylate) microcapsules, respectively, or in colloidal drug delivery systems, for example, liposomes, albumin microspheres, microemulsions, nanoparticles, and nanocapsules or in macroemulsions. Such teachings are disclosed in Remington's Pharmaceutical Sciences (1980), which is incorporated herein by reference.

Examples of suitable aqueous and non-aqueous carriers which may be employed in the pharmaceutical composition of the present invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

It is possible to enhance the biological half-life or bioavailability of the Plac9 or its derivatives of the present invention, thereby increasing the retention or stability of the derivatives in a recipient, by bonding which to one or more chemical “moieties’ in order to produce a compound which can be recognized and processed within a recipient to yield a PLAC9 derivative. The “moieties’ for bonding to the present PLAC9 may include one or more lipids, carbohydrates, amino acid residues, etc. Preferably, a preferred “moiety’ is an amino acid residue or nucleic acid. More preferably, the “moiety’ is a peptide or oligonucleotide. The amino terminal residue of PLAC9 is a preferred site for the bonding of the “moiety.”

The composition or pharmaceutical composition of the present invention may also contain adjuvants, such as preservatives; wetting agents, emulsifying agents and dispersing agents such as sodium lauryl sulfate and magnesium stearate; as well as coloring agents, release agents, coating agents, sweetening, flavoring and perfuming agents, preservatives, solubilizing agents, buffers and antioxidants. Prevention of the action of microorganisms upon the compounds of the present disclosure may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

To determine functional attributes and efficacy of the present protein and its derivatives, various assays are employed to test the present protein and its derivatives in different in vitro and in vivo models including glucose uptake by adipocytes or insulin secretion measurement from various pancreatic cells/tissues, physiological change and glucose/insulin/pyruvate tolerance tests in high fat diet-induced obese mouse model, streptozotocin-induced diabetic mouse model, etc.

The following examples accompanied with corresponding drawings are intended to better illustrate various embodiments of the present invention. Scope of the present invention should be defined in the appended claims.

### EXAMPLES

### Example 1—Improvement in Glucose Tolerance by Recombinant Plac9 in Diet-Induced Diabetic Model

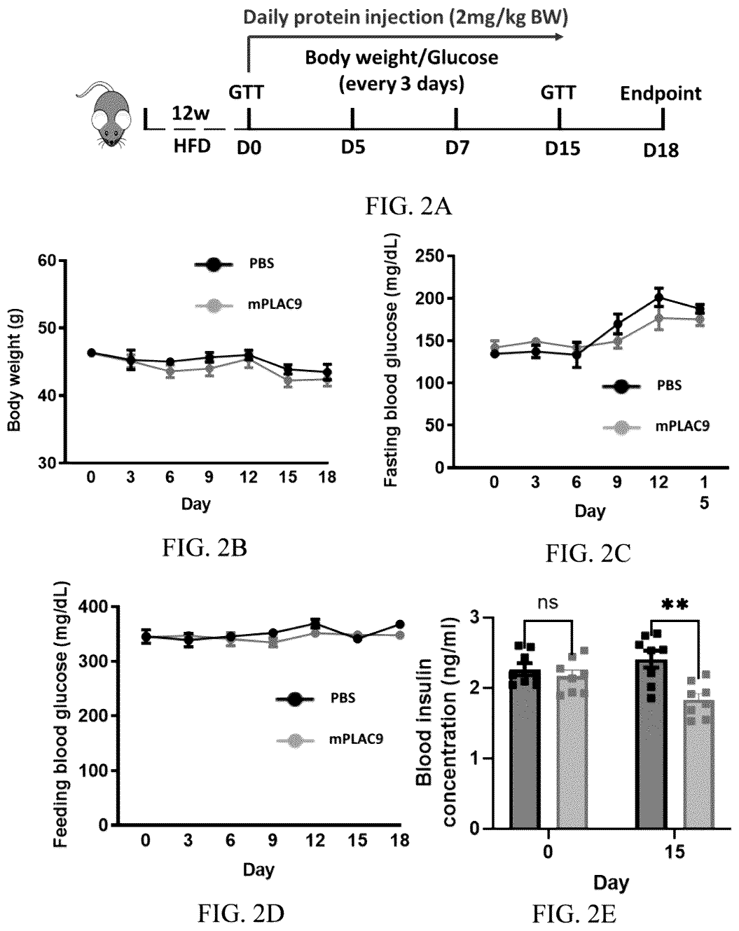

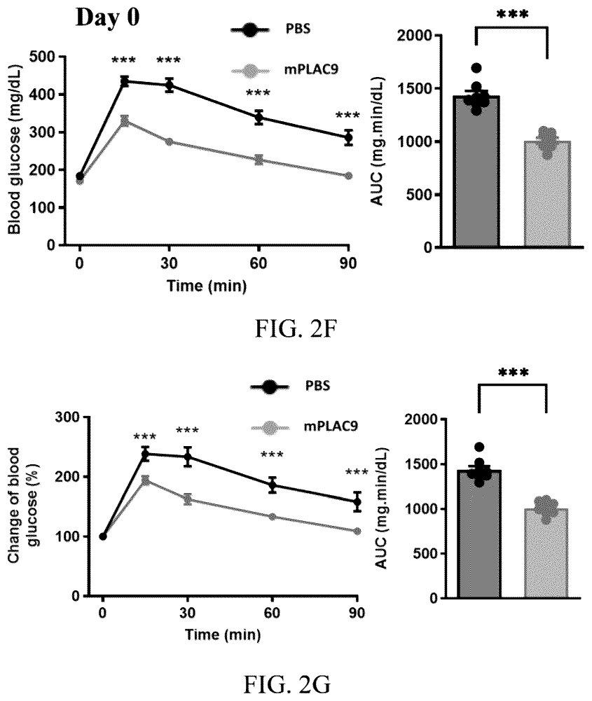

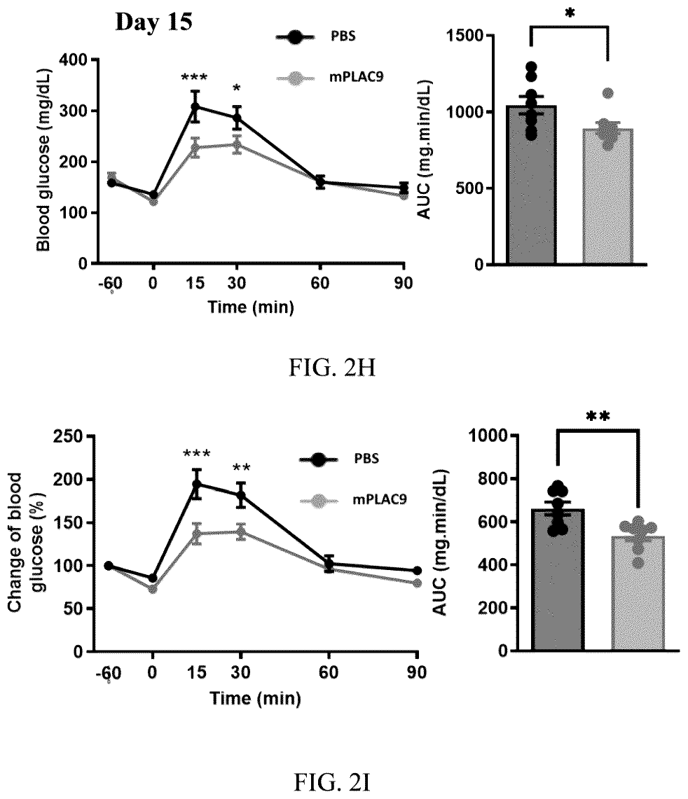

Turning to FIG. 2A, 8-week-old male C57BL/6J mice (n=8) were fed with a high-fat diet (HFD) for 12 weeks to induce a diabetes mellitus type 2 (DM2) model and then treated with 2 mg/kg (per body weight; hereinafter as “BW”) of recombinant mouse Plac9 (synthesized and purified from E. coli expressing mouse Plac9 represented by SEQ ID NO: 2; hereinafter as “mPLAC9”) once daily via intraperitoneal (i.p.) injection for 18 days to evaluate its effects on glucose uptake and tolerance in an obese, diet-induced diabetic model. Body weight (FIG. 2B), fasting blood glucose level (FIG. 2C), feeding blood glucose level (FIG. 2D) and blood insulin level (FIG. 2E) were measured at the beginning of the i.p. injection and then once every 3 days until the end of the experiment (i.e., Day 3, 6, 9, 12, 15 and 18). Another group of HFD-fed mice was injected with PBS once daily and measured with the same biometric data as in the Plac9-injected group (treatment group) once every 3 days from the beginning of the injection until the end of the experiment.

As seen from FIG. 2B, both treatment and control groups had similar body weight change throughout the whole experiment, and no obvious weight change was observed in the treatment group. Similarly, no obvious changes in fasting and feeding blood glucose levels were observed in the treatment group as compared to those of control group (FIGS. 2C-2D). The most obvious change was in the blood insulin level in the treatment group compared with the control group (FIG. 2E), where the blood insulin concentration was reduced in the treatment group at Day 15.

Glucose tolerance test (GTT) was also performed in both the treatment and control groups at Day 0 and Day 15 of the experiment. The results are shown in FIGS. 2F-2I. As seen from FIGS. 2F-2G, the blood glucose level in the treatment group was more significantly reduced compared with the control group over a 90-minute time course at Day 0. At Day 15, the overall blood glucose level was also lower in the treatment group than that of the control group over the 90-minute time course (FIGS. 2H-2I).

From the above results, it is suggested that Plac9 can improve the glucose tolerance in an obese, diet-induced diabetic model. It is also expected that human Plac9 (represented by SEQ ID NO: 1) having a sequence homology of about 73% similarity to that of mouse Plac9 (represented by SEQ ID NO: 2) may also have similar beneficial effect on improving glucose tolerance in an obese, diabetic (type 2 diabetes) human subject.

### Example 2—Improvement in Glucose Tolerance by Recombinant Plac9 in Insulin-Deficient Diabetic Model

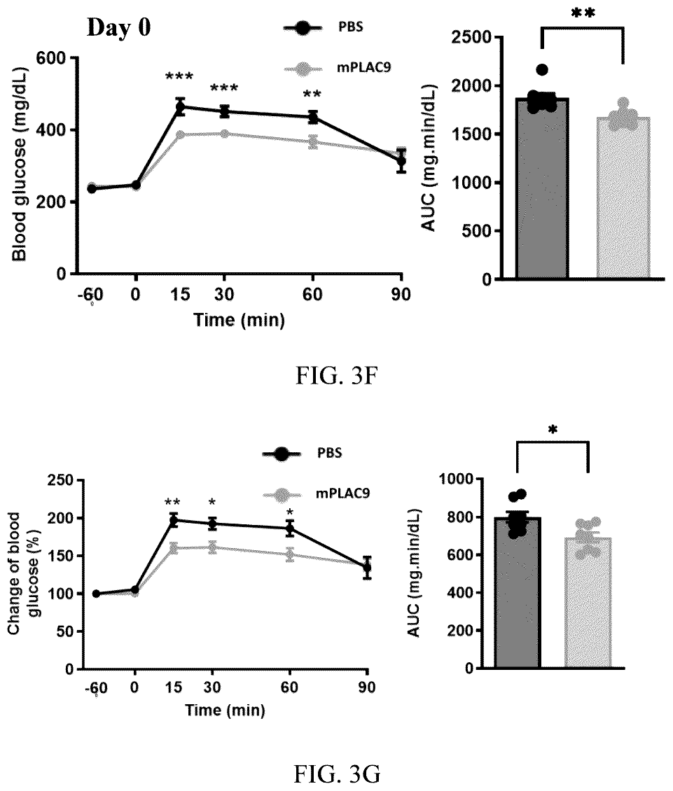

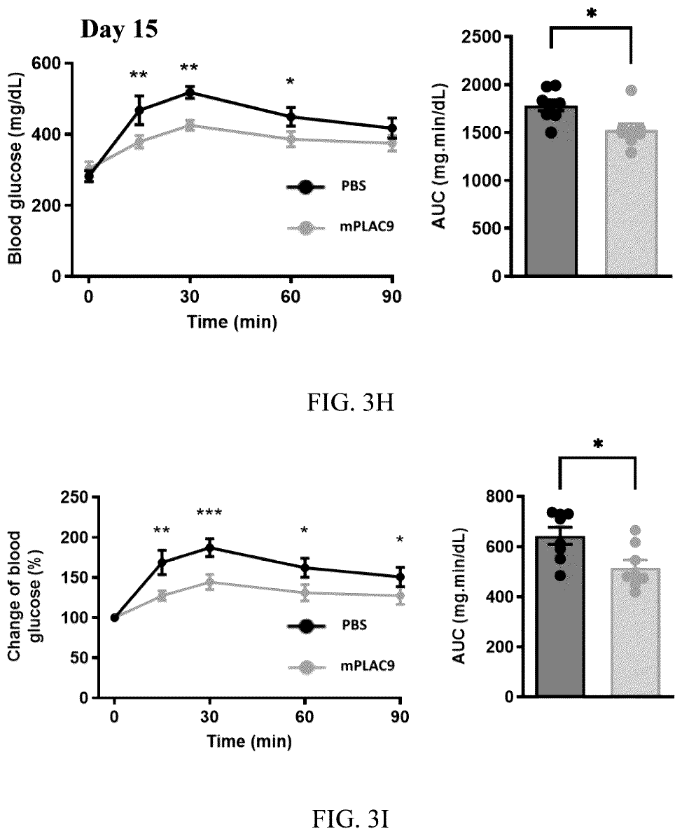

Turning to FIG. 3A, 8-week-old male C57BL/6J mice (n=8) which were fed with standard chow (STC) diet were treated with streptozotocin (STZ) for 5 days to induce diabetes. Similar to Example 1, 2 mg/kg (BW) of recombinant mPLAC9 was administered to the STZ-induced diabetic mice via i.p. injection once daily for 18 days (treatment group). In parallel, PBS was injected to STZ-induced diabetic mice for 18 days as a control group. Same biometric data as in Example 1 were measured once every 3 days, and the results are shown in FIGS. 3B-3E, respectively. As seen from FIGS. 3B-3D, changes in body weight, fasting glucose and feeding glucose levels were similar between the treatment and control groups throughput the experiment. The main difference between the two groups was observed in blood insulin level. As seen from FIG. 3E, the blood insulin levels between the two groups were similar from Day 0 to Day 5. Interestingly, staring from Day 5, the blood insulin concentration was increased in the treatment group till the end of the experiment, but the blood insulin level could not be restored to the level measured at Day 0, whereas there was no change in the insulin level in the control group since Day 5.

GTT was also performed at Day 5 and Day 15 in this STZ-induced diabetic model. As seen from FIGS. 3F-3G, the overall blood glucose level in the treatment group throughout the 90-minute time course at Day 0 was lower than that in the control group. Similar trend was observed between the treatment and control groups at Day 15 (FIGS. 3H-3I).

From the above results, it is suggested that Plac9 can improve glucose tolerance in an insulin-deficient diabetic model. It can also be contemplated that the glucose uptake induced by Plac9 is in an insulin-independent manner. Furthermore, it is suggested that Plac9 can also increase an endogenous insulin level. Further study as to the role of Plac9 in regulating glucose metabolism and whether there is any synergistic effect when co-administered with insulin is required.

### Example 3—Glucose Metabolism Regulation by AAV-mediated Plac9 Overexpression in Diet-Induce Diabetic Model

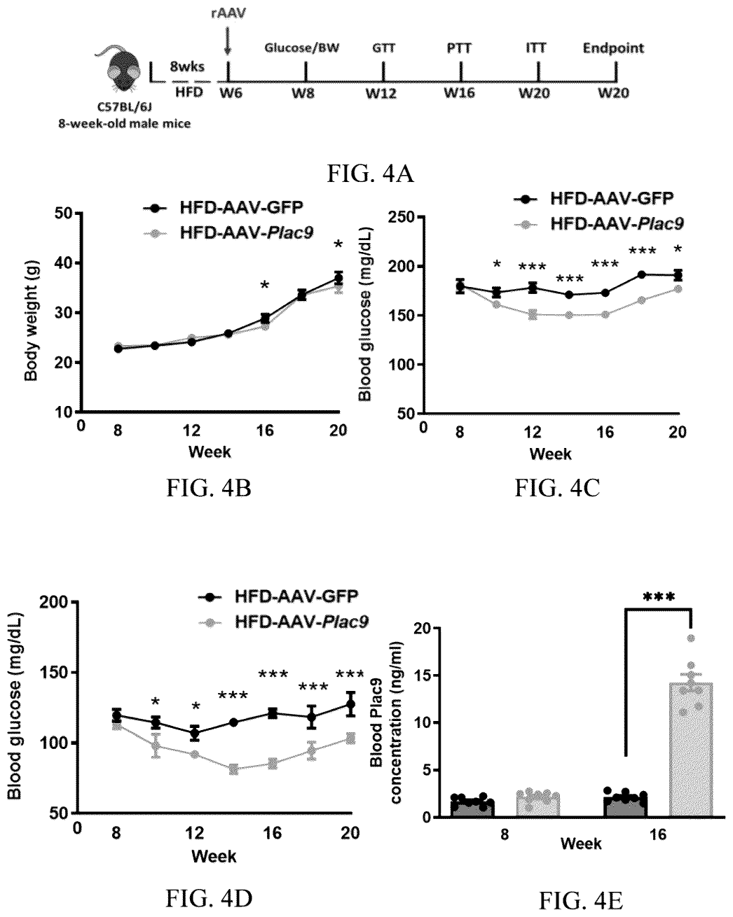

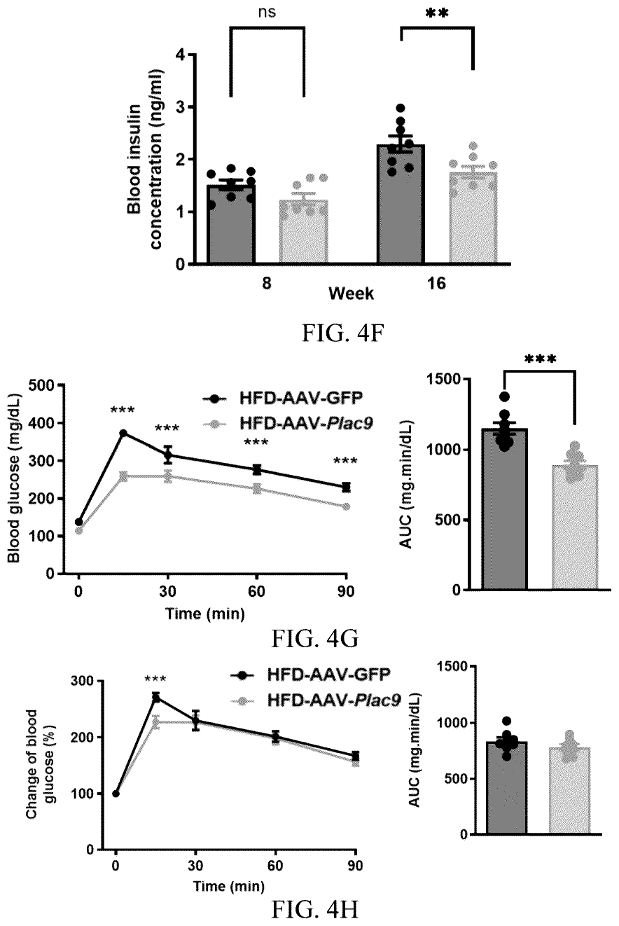

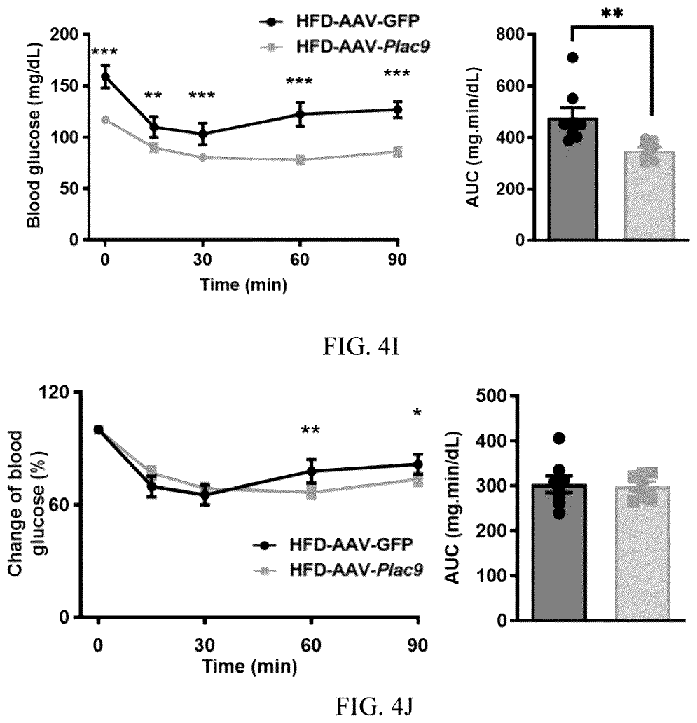

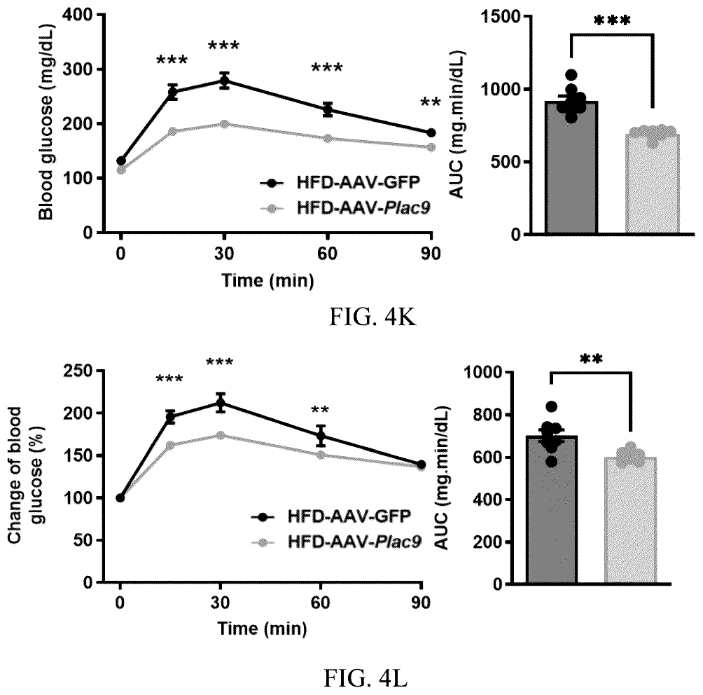

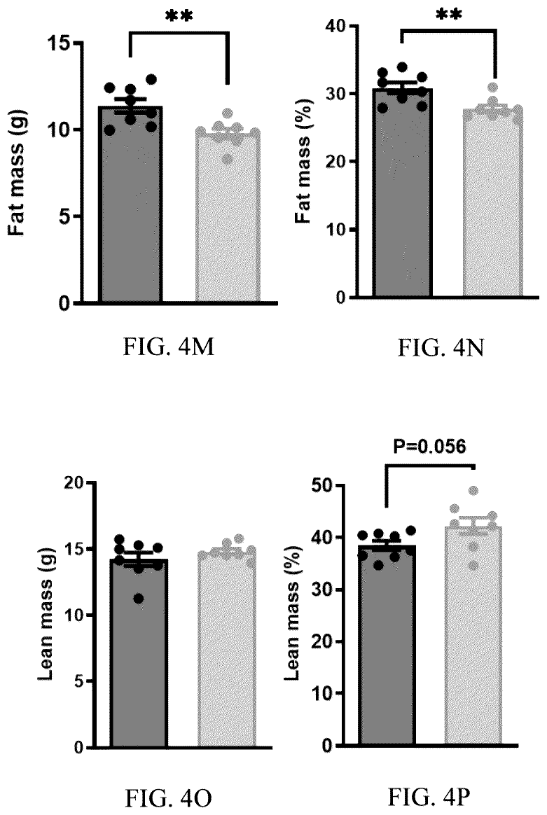

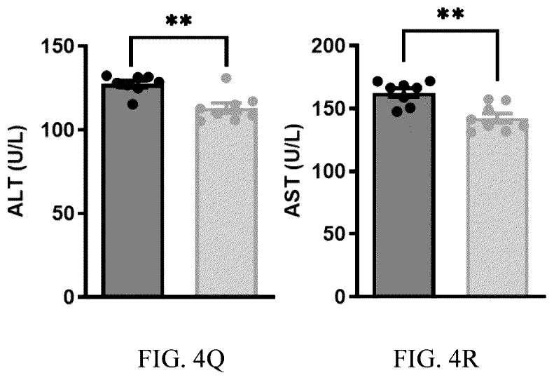

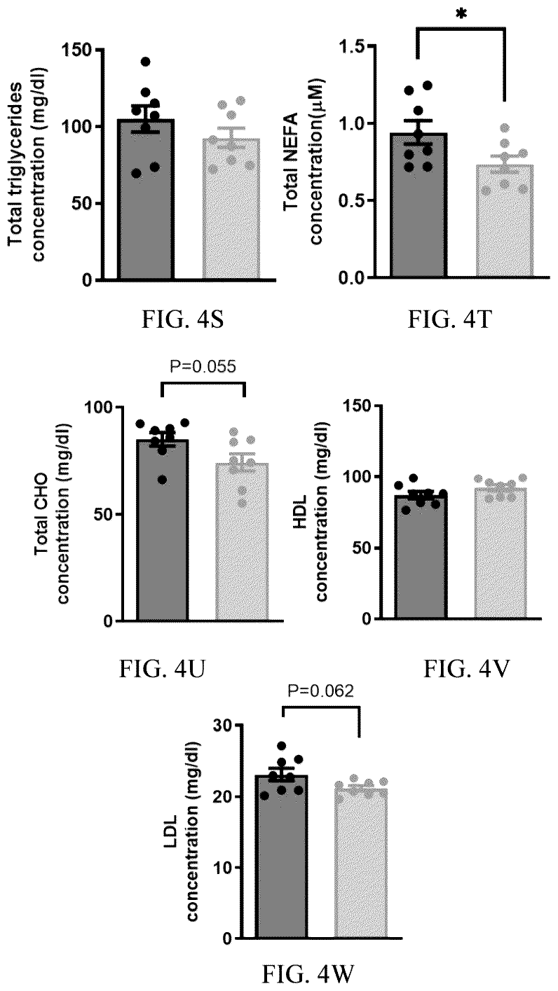

Turning to FIG. 4A, an HFD-induced diabetic model was provided in 8-week-old male C57BL/6J mice (n=8) and infected with an adenovirus (AAV) overexpressing Plac9 (Adeno-Plac9) via intravenous (i.v.) injection at Week 6 (treatment group) to study the role of Plac9 in glucose metabolism in vivo. Another group of HFD-fed mice were treated with an AAV overexpressing green fluorescence protein (Adeno-GFP) only (control group). Similar to the two foregoing examples, biometric data such as body weight (FIG. 4B), fasting and feeding glucose levels (FIGS. 4C and 4D) once every 2 weeks starting from Week 8 till the end of the experiment, and other biometric data such as serum (circulating) Plac9 level (FIG. 4E) and circulating insulin level (FIG. 4F) at Week 8 and Week 16, were measured. In FIG. 4B, no significant difference was observed in body weight between the two groups. However, the fasting and feeding glucose levels were reduced more significantly in the first 4 to 6 weeks after i.v. injection of Adeno-Plac9 in the treatment group than the control group injected with Adeno-GFP (FIGS. 4C and 4D). Overall, the treatment group had lower circulating blood glucose levels throughout the experiment than those of the control group. Interestingly, the increase in circulating Plac9 concentration between Week 8 and Week 16 was much more significant than the circulating insulin concentration in the treatment group, as compared to the control group where the circulating insulin level and the increase in the insulin concentration from Week 8 to Week 16 were both higher than those of the treatment group (FIGS. 4E and 4F), suggesting that Plac9 is not positively correlated with the insulin level secreted by the pancreatic beta cells. The above results further suggest that Plac9 can reduce the blood glucose level in a diet-induced diabetic model in an insulin-independent manner.

Similar to the two foregoing examples, GTT was also performed in this Adeno-Plac9-treated diet-induced diabetic model at Week 12, and the results are shown in FIGS. 4G-4H. Insulin tolerance test (ITT) and pyruvate tolerance test (PTT) were performed at Week 20 and Week 16, respectively, and the results are shown in FIGS. 4I-4J and 4K-4L, respectively. As seen from FIGS. 4G-4H, an improved glucose homeostasis was observed in the treatment group compared with the control group. FIGS. 4I-4J reveals that insulin sensitivity in the treatment group was not improved by the Adeno-Plac9 as the level of and the change in blood glucose level in the treatment group was similar to those in the control group. The lower blood glucose level in the treatment group in response to a bolus of pyruvate than that in the control group suggests that Plac9 may play a role in regulating hepatic gluconeogenesis (FIGS. 4K-4L).

FIGS. 4M-4P show the difference in fat mass and lean mass before and after the experiment in the treatment and control groups, and the results suggest that Plac9 overexpression promotes tissue, muscle and bone growth rather than fat in the obese, diet-induced diabetic model, as compared to the control group. FIGS. 4Q-4X demonstrate that Plac9 overexpression is able to prevent liver damage or failure from high blood glucose level by lowering triglycerides (TG), free fatty acids (NEFA), cholesterols (CHO) and low density lipoprotein (LDL) levels while increasing the high density lipoprotein (HDL) level in the diet-induced diabetic model.

Overall, it is suggested that Plac9 has positive effects on improving glucose uptake in an insulin-independent manner and maintaining glucose homeostasis through liver.

### Example 4—Insulin-Independent Glucose Uptake Stimulated by Plac9 in Liver Cells or Hepatocytes

To verify that the glucose homeostasis is regulated through liver or hepatocytes by overexpression of Plac9 in an insulin-independent manner, an in vitro insulin secretion assay was performed on confluent pancreatic beta cells, MIN6, in each well treated by 100 nm of Exendin-4 or different concentrations of recombinant Plac9 of mouse origin at 10, 100 and 1000 nM (n=8) and measured the insulin content of each well by a mouse insulin ELISA kit. The results in FIG. 5A suggest that Plac9 does not induce insulin secretion at all concentrations tested, compared to 100 nM of Exendin-4 which significantly induces insulin secretion by the MIN6 cells. A glucose uptake assay was performed on primary hepatocytes and hepatic cell line, HepG2, treated with different concentrations (from 0.125 or 0.25 to 1.0 μM) in the presence or absence of exogenous 10 nM insulin, and the results are shown in FIGS. 5B and 5C, respectively. In FIG. 5B, the glucose uptake in the primary hepatocytes enhanced by Plac9 is in a dose-dependent manner, with or without co-administration of insulin. Similar trend was observed in HepG2 cells (FIG. 5C). Overall, the glucose uptake induced by Plac9 in liver or hepatocytes is verified to be in an insulin-independent manner, and co-administration with the insulin can further enhance the glucose uptake, thereby lowering the demand for or reliance on insulin.

Although the invention has been described in terms of certain embodiments, other embodiments apparent to those of ordinary skill in the art are also within the scope of this invention. Accordingly, the scope of the invention is intended to be defined only by the claims which follow.

What is claimed is:

1. A compound capable of lowering circulating glucose level in an insulin-independent manner comprising a protein, peptide, or any derivative thereof.

2. The compound of claim 1, wherein the protein or peptide is represented by an amino acid sequence of SEQ ID NO: 1 or 2.

3. The compound of claim 1, wherein the protein is a recombinant placenta-specific protein 9 (Plac9).

4. The compound of claim 3, wherein the recombinant Plac9 is synthesized and purified from a bacterial expression system.

5. The compound of claim 1, wherein the derivative is a protein or peptide having an amino acid sequence of SEQ ID NO: 3 or 4, or a protein or peptide with a sequence homology of at least 73% similarity to the amino acid sequence of SEQ ID NO: 1 or 2, or a nucleotide encoding any of said protein or peptide.

6. A composition comprising an effective amount of the compound or the derivative thereof according to claim 1.

7. The composition of claim 6, wherein the composition further comprises a pharmaceutically acceptable carrier, ester, or salt.

8. A method for lowering circulating glucose level in a subject comprising administering the composition according to claim 6 to a subject in need thereof.

9. The method of claim 7, wherein the composition is administered via intravenous, intraperitoneal, or intramuscular injection, or via an oral or subcutaneous route.

10. The method of claim 7, wherein the composition further comprises a pharmaceutically acceptable carrier, ester, or salt.

11. The method of claim 7, wherein the subject comprises human or non-human mammals.

12. A method for treating diseases or conditions associated with or arising from hyperglycemia, glucose intolerance, or pancreatic beta cell dysfunction comprising administering the composition according to claim 6 to a subject in need thereof.

13. The method of claim 12, wherein the composition is administered via intravenous, intraperitoneal, or intramuscular injection, oral or subcutaneous administration.

14. The method of claim 12, wherein the composition further comprises a pharmaceutically acceptable carrier, ester, or salt.

15. The method of claim 12, wherein the subject comprises human or non-human mammals.

16. The method of claim 12, wherein the diseases or conditions comprise diabetes mellitus comprising type 1 and type 2 diabetes, cardiovascular diseases comprising hypertension, heart attack, and stroke, blindness, kidney failure, and lower limb amputation.


SEQUENCE LISTING (6 sequences)

SEQ ID NO: 1 - protein, 75 aa, Homo sapiens
AEPFSPPRGDSAQSTACDRHMAVQRRLDVMEEMVEKTVDHLGTEVKGLLGLLEELAWNLPPGPFSPAPDLLGDGF

SEQ ID NO: 2 - protein, 89 aa, Mus musculus
EWGQGPRDTPGRRAAESPSSPGDLAGSPGCDRHAAVQRRLDIMEETVEKTVEHLEAEVTGLLGLLEELASNLPTGPFSPKPDLLGDDGF

SEQ ID NO: 3 - protein, 87 aa, Homo sapiens
MRPLLCALTGLALLRAAGSLAAAEPFSPPRGDSAQSTACDRHMAVQRRLDVMEEMASEPWSWSPAVGGGAPARQRPQNQPCPLDFLP

SEQ ID NO: 4 - protein, 55 aa, Homo sapiens
MAVQRRLDVMEEMVEKTVDHLGTEVKGLLGLLEELAWNLPPGPFSPAPDLLGDGF

SEQ ID NO: 5 - protein, 97 aa, Homo sapiens
  SIGNAL 1..22: note = signal peptide
  CHAIN 23..97: note = chain region
MRPLLCALTGLALLRAAGSLAAAEPFSPPRGDSAQSTACDRHMAVQRRLDVMEEMVEKTVDHLGTEVKGLLGLLEELAWNLPPGPFSPAPDLLGDGF

SEQ ID NO: 6 - protein, 108 aa, Mus musculus
  CHAIN 20..108: note = chain region
  SIGNAL 1..19: note = signal peptide
MQALLCALAGLALLRAGTGEWGQGPRDTPGRRAAESPSSPGDLAGSPGCDRHAAVQRRLDIMEETVEKTVEHLEAEVTGLLGLLEELASNLPTGPFSPKPDLLGDDGF